(19) United States
(12) Patent Application Publication
(10) Pub. No.: US 2019/0233467 A1
(43) Pub. Date: Aug. 1, 2019

(54) REACTION CELL FOR ASYNCHRONIOUSMULTIPEPTIDE INSTRUMENT

(71) Applicant: Vasiliy Abramov, Louisville, KY (US)

(72) Inventor: Vasiliy Abramov, Louisville, KY (US)

(21) Appl. No.: 16/299,954

(22) Filed: Mar. 12, 2019

Related U.S. Application Data
(63) Continuation-in-part of application No. 15/409,048, filed on Jan. 18, 2017.

(51) Int. Cl. C07K 1/04; B01J 19/00

(52) U.S. Cl. CPC C07K 1/045; B01J 2219/00686; B01J 2219/00164; B01J 19/0046

(57) ABSTRACT

A reaction cell for an automated peptide synthesizer consists of a body having adjacent first and second reaction wells for simultaneous reactions. The first reaction well is in fluid communication with the second reaction well for reagent pre-activation simultaneously with an amino acid addition for solid state peptide production.

### FIELD OF THE INVENTION

This invention relates to the synthesis of peptides and more particularly to automated peptide synthesis instruments.

### BACKGROUND OF THE INVENTION

In the synthesis of peptides by solid state peptide synthesis (SPPS) automated peptide synthesis instruments provide substantial labor and time savings in the solid-state synthesis of peptides. There are a variety of peptide instruments in the prior art, however, of particular interest is the TETRIS™ asynchronous multi-marginspeptide instrument distributed by Advanced Chemtech, Louisville, Ky. This instrument, described in patent publication 20070140925, comprises a carousel carrying a plurality of individual reaction cells for moving the cells between injection stations containing reagents and ancillary solvents for delivery to the reaction cells. The instrument further includes purge stations for removing liquids from the reaction cells upon completion of a synthesis step. The instrument is programmable to produce a variety of peptides.

### SUMMARY OF THE INVENTION

The present invention is designed fur use in automated chemical synthesizers. Depending on the peptide being synthesized, there may be an activation step to activate the particular reagents for the next reaction. As pointed out. above for purposes of description the invention will be described in connection with the TETRIS™ peptide synthesizer although, with modifications to the instrument, the reaction cell can be employed on other types of automated instruments.

With conventional reaction cells for the TETRIS™ the cell is moved into alignment with an injection station containing the activation reagent. Following the activation step the cell is moved to an injection station to receive the next amino acid to be coupled according to the selected protocol. in other types of peptide synthesizers, the reagents may be brought to the reaction cell rather than moving the reaction cell to stationary injection stations.

The efficiency of the solid-state peptide synthesis is improved and time saved by the present invention comprising an improved dual well reaction cell in which activation of reagents and the reaction resulting in the addition of an amino acid to a reaction product on the substrate are carried out essentially simultaneously. In accordance with the invention a novel reaction cell is provided that comprises duel but separate wells that are in one-way fluid communication. In this manner a reaction step can be carried out simultaneously with an activation step so as to essentially combine two steps of a synthesis in a single cell. Both the activation step and the reaction step require some time in which to complete the steps. By carrying out both steps at essentially the same time the efficiency of the instrument is improved.

### BRIEF DESCRIPTION OF THE DRAWINGS

FIG. 1 is a perspective view of a preferred embodiment of the reaction cell of the invention;

FIG. 2 is a perspective view of FIG. 1 showing the interior of FIG. 1;

FIG. 3 is a top plan view of the reaction cell of FIG. 1;

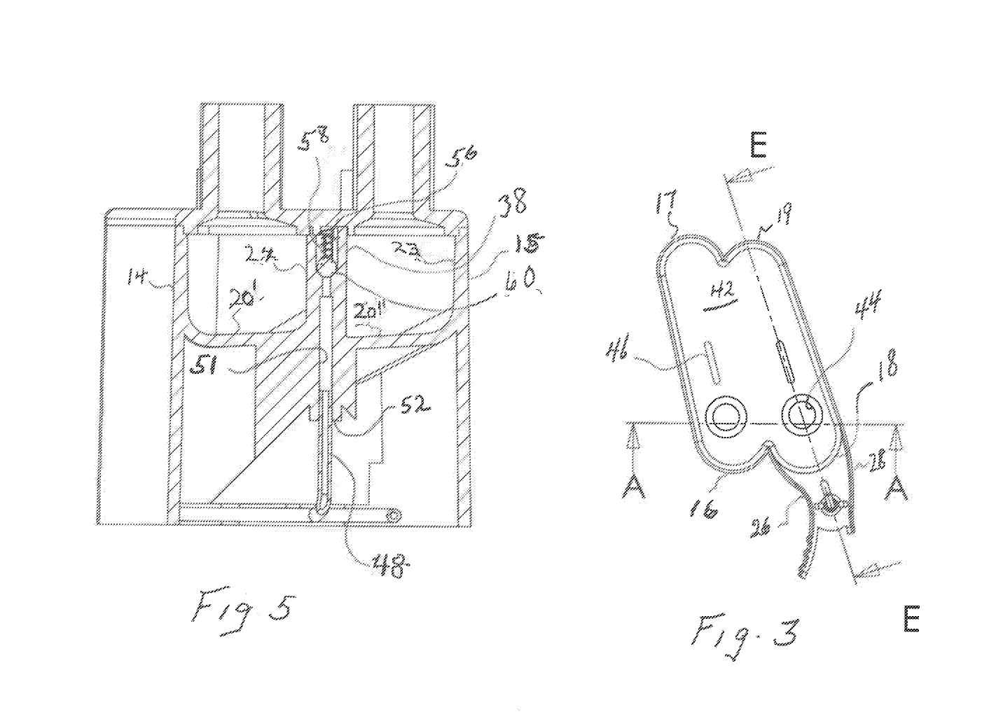

FIG. 4 is a bottom plan view of the reaction cell of FIG. 1;

FIG. 5 is an end sectional view of the reaction cell of FIG. 1;

### DESCRIPTION OF THE INVENTION

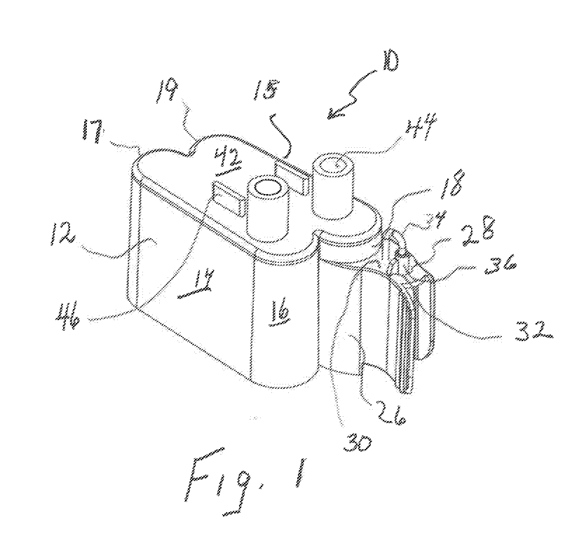

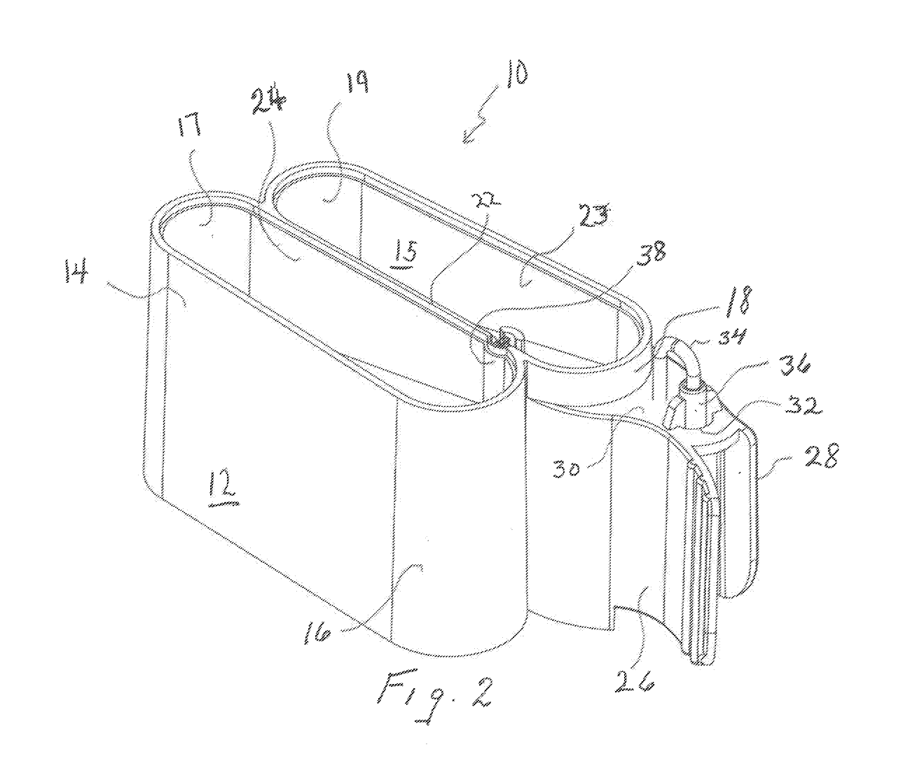

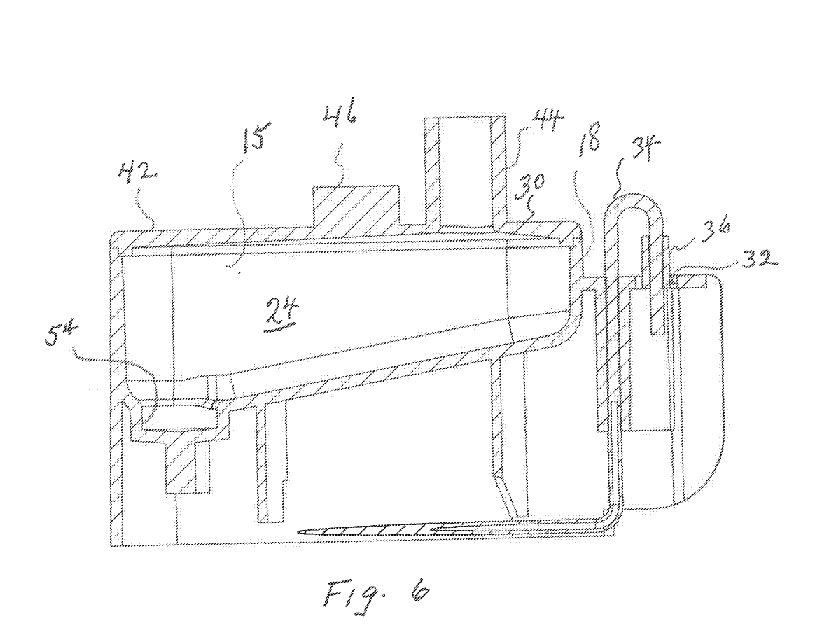

FIG. 1 and FIG. 2 illustrate a reaction cell shown generally as 10 comprising a body 12 having opposed sidewalls 14 and 15, end walls 16, 17, 18, and 19, bottom walls 20′ and 20″ and inner common wall 22 which cooperate to define a pre-activation well 23 and a reaction well 24. In the preferred embodiment adapted for solid state synthesis peptide production well 23 is used as a pre-activation well and well 24 for solid state peptide synthesis. The inner common wall 22 and side wall 15 are provided with extensions 26 and 28 and a transverse member 30 extends from the end wall 18 and between the extensions 26 and 28. A port 32 is provided for a discharge line 34 and a discharge line connector body 36 is provided for discharge of spent reagent from the well 24. An open-ended passage 51 in the common wall 22 defines a port 52 at one end for a fluid transfer line 48 and defines a valve body 38 for a spring-loaded valve 56 (FIG. 5) to provide one -way fluid communication between the well 23 and the well 24. The wells 23 and 24 are closed by a lid 42 having ports 44 for introduction of reagents to the wells. Vertically extending members 46 on the lid 42 serve as grips for handling the lid.

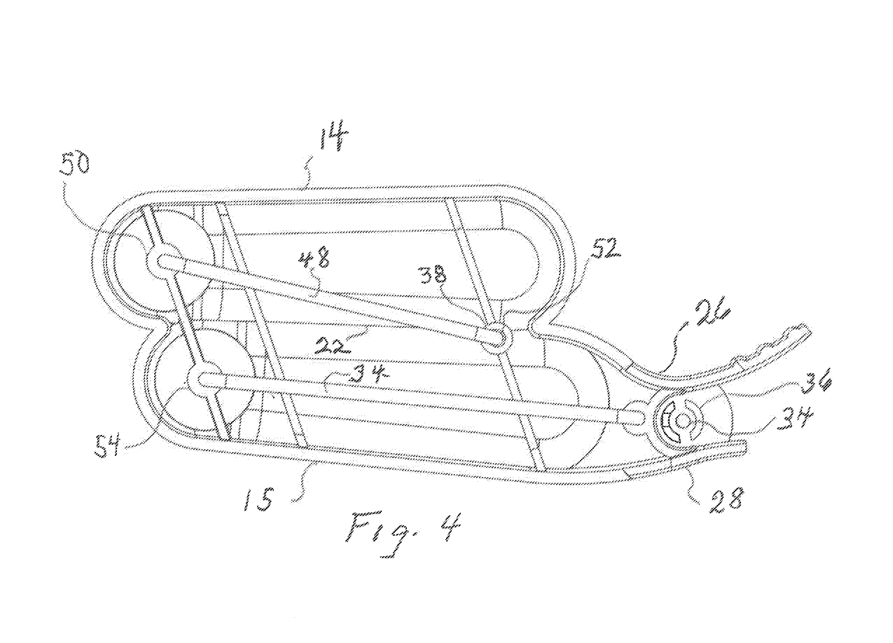

Referring to FIG. 4 the transfer line 48 provides fluid communication between port 50 and port 52 for transfer of pre-activated reagents from well 23 to well 24 through the passage 51. Discharge line 34 extends between port 54 and the discharge line connector 36 for discharge of spent reagents.

FIG. 5 more clearly illustrates the transfer line 48 communicating through the port 52 and passage 51 with a one-way valve 56 comprising a check valve body 60 seated in the valve body 38. The check valve body 60 is loaded by a spring 58 for one-way fluid communication between the well 23 and the well 24.

FIG. 6 most dearly shows the discharge line 34 and the discharge line connector 36 for discharging spent reagent through the port 32 to waste.

From the foregoing it can be seen that a pre-activation step can be carried out concurrently with the peptide synthesis so that the efficiency of a peptide synthesizer instrument is improved by the reaction cell of the present invention.

Having defined the invention,

I claim:

1. A reaction cell for an automated peptide synthesizer, said reaction well comprising a body front, back and side walls defining an interior, a common wall in said interior extending between said front and said back walls to divide said interior into two reaction wells, an open ended passage in said common wall, one open end defining a port for a fluid transfer line communicating from one of said reaction wells to said port, one other opposite open end of said passage communicating with said other reaction well, a valve body formed at said other opposite end of said passage, a check valve comprising a spring loaded valve body disposed in said valve body for one-way fluid communication between said reaction wells.

2. The reaction cell of claim 1 wherein one said reaction wells is a pre-activation well and said other reaction well is a peptide synthesis well whereby in a solid-state peptide synthesis protocol a pre-activation step and a peptide synthesis reaction can be carried out simultaneousl
